(19) United States
(12) Patent Application Publication
(10) Pub. No.: US 2014/0342380 A1
(43) Pub. Date: Nov. 20, 2014

(54) VERIFYING THE SOURCE OF BIOLOGICAL SAMPLES; METHOD, COMPOSITION AND KIT THEREFOR

(71) Applicant: Daniel Saal, Palo Alto, CA (US)

(72) Inventor: Daniel Saal, Palo Alto, CA (US)

(21) Appl. No.: 14/359,845

(22) PCT Filed: Nov. 21, 2012
(86) PCT No.: PCT/US2012/066384

§ 371 Date: May 21, 2014

Related U.S. Application Data
(60) Provisional application No. 61/562,351, filed on Nov. 21, 2011.

(51) Int. Cl. G01N 33/94; G06F 17/30

(52) U.S. Cl. CPC G01N 33/94; G06F 17/30289
USPC 435/7.92; 436/501; 707/736

(57) ABSTRACT

The present invention relates to a method, composition and a kit for verifying the source of biological samples when collected for testing, e.g., for the presence of drugs of abuse. The method generally involves providing a subject with a composition comprising at least one detectable consumable marker and optionally at least one metabolizable detectable consumable marker; obtaining a biological sample from the subject after consumption of the composition; and detecting the presence or absence of the detectable consumable marker in the sample; wherein the presence of the detectable marker indicates that the subject is the source of the sample, and wherein the absence of the detectable marker indicates that the subject is not the source of the biological sample.

### CROSS-REFERENCE

This application claims the benefit of U.S. Provisional Application No. 61/562,351 filed Nov. 21, 2011, which application is incorporated herein by reference.

### FIELD OF INVENTION

The present invention relates generally to analysis of a biological sample, e.g., particularly for verifying the source of the biological sample.

### BACKGROUND OF THE INVENTION

There are a number of relevant settings in which the determination and/or confirmation of the source of a biological sample is important. In clinical settings, toxicology is part of the standard evaluation of patients, e.g., on long-term opiate medication. Typically, a primary care clinician will order a test and the patient will go to a laboratory either associated with or separate from the clinic site for sample collection and processing. These collection sites are typically used for collection of all types of urine samples. As such, there is currently no mechanism for verifying that the sample is from the patient in question.

A second clinical setting is that of the drug treatment center. In most drug treatment centers toxicology is a routine part of the treatment regimen. Monitoring toxicology allows the drug treatment professional to ascertain the status of the subject and insure that those subjects who are intoxicated do not enter the treatment community. In addition, there are patients who have been sent by their workplace or by the courts for drug abuse treatment, and these patients are not necessarily motivated to abstain from using drugs or alcohol and may use banned substances. The types of testing centers that perform drug testing, e.g., drug treatment centers or drug courts, often supervise or observe the subject as he is giving a urine sample. This is usually done by watching through a one-way mirror or by entering the stall with the patient. This is very uncomfortable for both the subject and the observing clinician and therefore is not commonly done, even when appropriate.

Outside of the clinical setting, there are a number of jobs that require initial or ongoing drug testing. These include positions involving mass transit and other areas in which the safety of others is involved. In addition, athletes are often required to test for drugs or other banned substances. In these settings the presumption is that the subject is not using drugs, and accordingly this makes it impractical to have sample collection be directly observed.

Another area of interest for urine toxicology is the legal system. There are a growing number of drug courts which are used as an alternative to the standard criminal court system. In non-violent drug offense cases, suspects can submit to the oversight of the drug courts and are required to do routine drug testing.

Another area of interest for urine toxicology is the military. Soldiers and support personnel in critical roles are periodically drug tested. Positive urine tests can result in discipline, discharge, or change of duty. Because of this, there is a high level of incentive to submit a false sample.

### SUMMARY OF INVENTION

Disclosed herein, are methods of verifying the source of a biological sample, comprising: (a) obtaining a biological sample from a subject after said subject ingested or consumed, or should have ingested or consumed, a composition comprising at least one detectable consumable marker; and (b) detecting the presence, absence, or quantity of said one or more detectable consumable markers or a modified thereof in the biological sample. In some embodiments, the method further comprises verifying whether said subject is the source of said biological sample by comparing results detected in step (b) with expected post-consumption profile in such biological sample. In a preferred embodiment, said biological sample is urine. In a further embodiment, the method further comprises obtaining said biological sample from said subject at a time after said subject ingested or consumed, or should have ingested or consumed, the composition wherein at least one detectable consumable markers is present in said biological sample in a detectable amount, if said composition was consumed. In some embodiments, the obtaining is performed after less than 2 hours, 1 hour, 30 minutes, 20 minutes, or 15 minutes from the time of ingestion. The composition may comprise a set of at least 2, 3, 4, 5, 6, or 7 detectable consumable markers. In some embodiments, at least one of the detectable consumable markers is detected in the biological sample in its parental (unmodified) form. In some embodiments, at least one of the detectable consumable markers is detected in the biological sample in its modified form.

In some embodiments, the method further comprises comparing the presence or absence of the set of detectable consumable markers in the biological sample to the expected post-consumption profile in said biological sample of said set of detectable consumable markers, wherein a match between the presence, absence, or quantity of the detectable consumable markers or modified forms thereof in said biological sample and an expected post-consumption profile is indicative that said subject is the source of said biological sample, and a second assay is performed on said biological sample. In some embodiments, the second biological assay is a urine test. In some embodiments, the second biological assay is selected from the group consisting of prenatal testing, pregnancy testing, drug testing, hormone testing, or liver function testing.

In some embodiments, the methods disclosed herein further comprise assigning the at least one detectable consumable marker to the subject prior to said obtaining step.

In some embodiments, the methods disclosed herein further comprise determining the baseline level of the at least one detectable consumable marker in a biological sample from the subject, prior to obtaining said biological sample.

In some embodiments, the methods disclosed herein further comprise observing the subject consume or ingest the composition.

In further embodiments, in the methods disclosed herein, the presence, absence or quantity of said at least one detectable consumable marker is detected with an antibody specific for said at least one detectable consumable marker. In some embodiments, the antibody specific for said at least one detectable consumable marker is attached to a solid support.

In some embodiments, the presence or absence of said at least one detectable consumable marker is detected using a chromatography technique.

Also disclosed herein are compositions consisting essentially of two or more detectable consumable markers, wherein the detectable consumable markers are non-toxic and generally safe for consumption. In some embodiments, the detectable consumable markets are Generally Regarded as Safe (GRAS) compounds.

In some embodiments, disclosed herein are compositions comprising three to six GRAS compounds. In some embodiments, the compositions comprise three to five GRAS compounds. In some embodiments, the compositions comprise three to four GRAS compounds. In some embodiments, the GRAS compounds are selected from: dilauryl thiodipropionate; ethyl formate; ethyl methylphenylglycidate; formic acid; geraniol; geraniol acetate; glucono delta-lactone; glycoryrrhiza; 3,5-diiodosalycylic acid; musk ambrette; n-Butoxypolyoxyethylene polyoxypropylene glycol; canaga; caraway; hyssop; immortelle; linden flowers; lovage; maidenhair/gingko; naringin; n-Octanoic acid; opopanax; pennyroyal; pipsissewa leaves; St. John's Wort; serpentaria; tannic acid; tuberose; zedoary; alpha-amylase; aminoglycoside 3′phosphotransferase II; amylase from aspercillgus oryzae; gum karaya; guarana; papain; sterculia gum; and sterculia gum.

In a preferred embodiment, the compositions are formulated for oral administration.

In further embodiments, the compositions are formulated so that each of the compounds is provided at a dose detectable in urine within 2 hours, 1 hour, 30 minutes, 20 minutes, or 10 minutes. In some embodiments, at least one GRAS compound is metabolized after consumption or ingestion. In some embodiments, the GRAS compounds are not generally found in foodstuffs or pharmaceuticals.

Also disclosed herein are kits comprising a composition consisting essentially of two or more detectable consumable markers, or compositions comprising two or more, three or more, or four or more GRAS compounds. In some embodiments, the kit further comprises a list of the consumable markers in said composition. In some embodiments, the kit further comprises a barcode associated with the composition. In some embodiments, the kits further comprise additional consumable compositions, each with a different barcode.

Also disclosed herein is a system comprising a database linking one or more unique barcodes with a unique set of detectable consumable markers or detectable modified forms thereof. In some embodiments, the database is located inside a computer. In some embodiments, the computer is linked to the internet. In some embodiments, the computer is capable of sending information to a remote location. In some embodiments, the computer is programmed to specifically perform one or more of the following tasks: (1) input barcode info; (2) link a barcode with contents of a composition; (3) confirm authenticity of the source of a biological sample; (4) provide a report; (5) transmit any of the above over the internet; (6) input data from screening assays; and/or (7) track authenticity from a single source over a period of time.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies a kit according to the invention. Within the kit box is at least one packet containing a consumable marker composition tablet, wherein the packet has two copies of a bar code for identification of the composition and its expected post-consumption profile. The kit can optionally comprise “dipstick” style testing strips for different detectable consumable markers (with a reaction (R), testing (T), and control zone (C) for each detectable consumable marker). The kit also optionally comprises a guide for verifying the source of a biological sample.

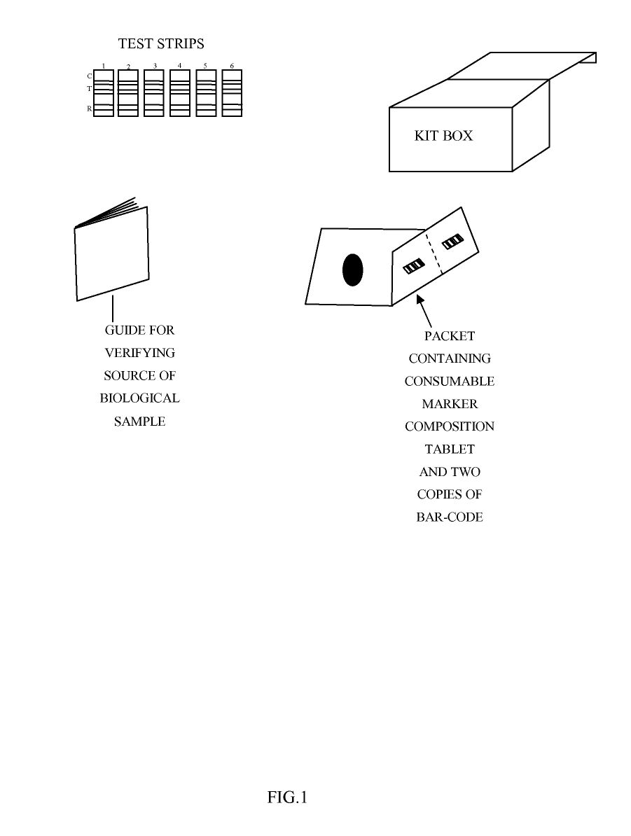

FIG. 2 exemplifies a system for verifying the source of a biological sample. A user submits a biological sample after consuming or ingesting a detectable marker composition, as disclosed herein. The sample is run through a detection apparatus comprising a solid support for antibodies specific for the detectable consumable markers within the composition and the detection apparatus determines whether the marker are present or absent, or the quantity of each marker in the sample. The detection apparatus is connected to a computer that compiles the information, and sends the information through the internet to the handheld device of an end user.

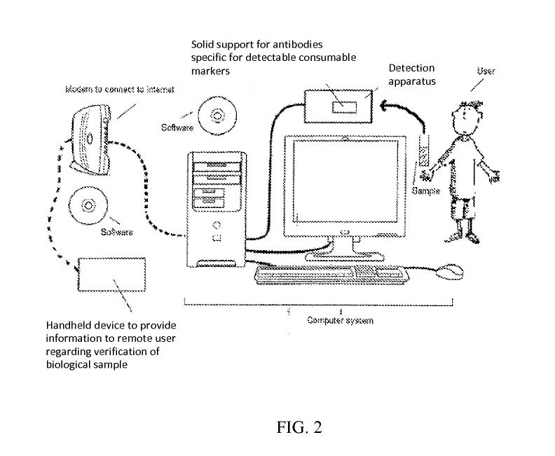

### DETAILED DESCRIPTION

Drug use and addiction are major problems in all aspects of life. The most common method of monitoring drug use by an individual is to collect a biological sample from the person, e.g., urine, and test it for the presence of drug, e.g., with immunological assays. Urine toxicology is a well-established technology. The greatest challenge to drug testing is insuring the authenticity and source of the sample.

There are a number of ways an individual may attempt to foil a standard drug test. The simplest is to dilute the sample with tap or toilet water. Individuals may also provide a biological sample from a different source. For example, drug-free (i.e., clean) urine samples may be purchased from any number of providers. Currently, when the source of a biological sample needs verification, collection of the biological sample from the subject needs to be witnessed. This level of discomfort discourages use of observed urine such that they are only used in the most extreme circumstances. Furthermore, even observed urine collections can be defeated by the use of a disposable reservoir with a clean sample (that may be artificial and/or purchased), which may be used in conjunction with a prosthetic penis or some other unisex device.

Accordingly, there is currently a need to assure the source and authenticity of a biological sample obtained from an individual. The present disclosure solves these problems by providing a method, composition, and kit that verifies the identity of a biological sample and eliminates possible attempts to foil a drug test while maintaining the privacy of an individual.

### DEFINITIONS

“Subject,” “patient,” and “individual” are used interchangeably and refer to, except where indicated, mammals such as humans and non-human primates, as well as rabbits, rats, mice, goats, pigs, and other mammalian species.

As used herein, the term “detectable” and “undetectable” refer to the detectability of a marker in a biological sample after it has been consumed, e.g., ingested, inhaled, administered sublingually, etc., by a subject. A detectable marker is either absorbed or unabsorbed.

As used herein, “biological sample” includes, but is not limited to, urine, feces, blood, saliva, cerebral spinal fluid, lymph fluid, sweat, hair, nails, and tissue biopsies. Such biological samples may be collected according to well-known methods using well-known collection devices. In a preferred embodiment, the biological sample is urine.

### Applications

For compounds that are absorbed in the digestive tract, there are a limited number of ways that the compounds can leave the body. The simplest is that the kidney may passively filter the compound into urine without modifying it. This is typically a relatively fast process taking from about several minutes to one or two hours from the time of ingestion. For some compounds, the compounds is chemically modified into a new compound, e.g., in the liver, and subsequently filtered or actively pumped into the urine. The method disclosed herein takes advantage of these known pathways of absorption and elimination of compounds by providing a composition comprising one or more compounds that are known to be present or absent in native or modified form, in a biological sample of interest after consumption by an individual.

Provided herein is a method by which the source of a biological sample, e.g., urine, may be reliably tracked. Generally, subjects who are to be tested are given a composition containing one or more compounds that are readily absorbed and detectable in the biological sample of interest. These compounds are referred to herein as “detectable consumable markers.” The detectable consumable markers may be detected either in parental (unmodified) or modified form after ingestion. A detectable consumable marker that can be detected either in parental or modified form after ingestion may serve as a positive control or positive standard for a biological sample. The composition can be taken orally, and compliance can readily be observed without discomfort to the observer or to the subject. When the individual provides the biological sample while the compounds are present in detectable amounts in the biological sample, the presence or absence of the compounds may be detected in the sample and indicate whether the sample came from the individual, and not from another person or from a water sample.

In some embodiments, a molecule or compound that is metabolizable in the body may be included in the composition as a detectable consumable marker, wherein the metabolizable compound is not expected to be present in a biological sample after ingestion. The presence of a metabolizable consumable marker compound in a biological sample would make a falsification attempt recognizable, i.e., if a subject spits the composition into a non-self urine sample, the presence of the metabolizable consumable marker in the sample would indicated that the composition was not ingested by the subject. Accordingly, a metabolizable consumable marker can function as a negative control or negative standard for a biological sample.

Accordingly, provided herein is a method of verifying the source of a biological sample collected for biochemical analysis, e.g., drug testing, prenatal testing, pregnancy testing, hormone testing, or liver function testing. In some embodiments, the biochemical analysis or assay is a urine test. In some embodiments, the method comprises: (a) obtaining a biological sample from a subject after said subject ingested or consumed, or should have ingested or consumed, a composition comprising at least one detectable consumable marker; and (b) detecting the presence, absence, or quantity of said one or more detectable consumable markers or a modified thereof in the biological sample. In some embodiments, the method further comprises verifying whether said subject is the source of the biological sample by comparing results in said detecting step with expected post-consumption profile in such biological sample. The presence of the at least one detectable consumable marker in the biological sample indicates that the subject is the source of the sample, and the absence of the detectable consumable marker indicates that the subject is not the source of the biological sample. To account for the possibility that the detectable marker may be present in foodstuffs or pharmaceuticals normally consumed by the individual, a baseline amount of the detectable marker may be established prior to consumption of the inventive composition. In some embodiments, the method further comprises observing the individual ingesting the composition. In some embodiments, the method further comprises assigning a composition comprising at least one detectable consumable marker to a particular subject prior to obtaining a biological sample from said subject.

In some embodiments, the method further comprises comparing the presence or absence of the set of detectable consumable markers in the biological sample to the expected post-consumption profile in said biological sample of said set of detectable consumable markers, wherein a match between the presence, absence, or quantity of the detectable consumable markers or modified forms thereof in said biological sample and an expected post-consumption profile is indicative that said subject is the source of said biological sample, and a second assay is performed on said biological sample. In some embodiments, the second biological assay is a urine test. In some embodiments, the second biological assay is selected from the group consisting of prenatal testing, pregnancy testing, drug testing, hormone testing, or liver function testing. In some embodiments, the results of the second biological assay are confirmed via an additional assay method, e.g., a positive drug test may be double checked by HPLC for the presence of drugs, or modified forms thereof.

In some embodiments, the ratios of the detectable consumable markers in a retrieved biological sample will be similar to the ratios of the detectable consumable markers in the composition. In some embodiments, the ratios of the detectable consumable markers in a retrieved biological sample will vary from the ratios of the detectable consumable markers in the composition.

The disclosed methods are useful for verifying the source of any biological sample. Typically, the methods disclosed herein would be employed to verify the source of a biological sample chosen from blood, urine, and cerebral spinal fluid. In a preferred embodiment, the biological sample is urine.

The detectable consumable markers used in the methods and compositions disclosed herein may be naturally occurring and/or synthetic molecules. The detectable consumable markers are generally non-toxic and safe for consumption. In some embodiments, the detectable consumable markers are derived from the list of Generally Recognized as Safe (GRAS) compounds generated by the United States Food and Drug Administration. One example of a GRAS compound appropriate for use as a detectable consumable marker in the subject invention is inulin, which is derived from the root of the chicory plant and has been used for various medicinal purposes due to its characteristic of being readily passively filtered by the kidney, e.g., detected in the urine after consumption. It is also not commonly found in most foods or pharmaceutical compositions.

Any number of other compounds can be selected, e.g., from the GRAS list, on the basis of their absorption and filtration or metabolic modification prior to clearance from the body. An exemplary list of suitable compounds finding advantageous use as detectable consumable markers is provided in Table 1 herein.

An example of a class of molecules that can be employed as metabolizable detectable consumable markers in the inventive methods and compositions is a peptide or protein. Proteins are broken down in the stomach and small intestines prior to absorption. Accordingly, post-ingestion, a protein will generally not be found in most biological samples, including urine, in its parental form. Accordingly, these metabolizable consumable markers can function as a negative control that would be expected to be absent if a subject has actually ingested a composition of detectable consumable markers including such a marker. An exemplary list of suitable compounds finding advantageous use as metabolizable consumable markers is provided in Table 2 herein.

In some embodiments, at least one detectable consumable marker is employed in the methods and compositions disclosed herein. In some embodiments, at least two, at least three, at least four, at least five, at least six, at least seven, at least eight, at least nine, at least ten, at least eleven, at least twelve, at least thirteen, at least fourteen, at least fifteen, or at least twenty detectable consumable markers are employed in the methods and compositions disclosed herein.

In instances whereby the markers in a composition disclosed herein are found in foodstuffs or pharmaceutical compositions, disclosed herein is a method to verify the source of a biological sample may further comprising determining the amounts of the detectable markers present in the biological sample and/or determining the baseline content of the marker in the biological sample prior to providing the subject with a composition disclosed herein. In some embodiments, subjects are provided with a list of foodstuffs and pharmaceutical compositions to avoid prior to consumption of a composition disclosed herein. In some embodiments, the falsification of a biological sample is avoided by the use of more obscure markers. In some embodiments, the components of a composition can be falsely represented to a subject in order to prevent subjects from obtaining clean urine and adding a cocktail of detectable consumable markers, e.g., compounds from the GRAS list, to falsely bypass this marker system.

Also disclosed herein is a system for verifying the source of a biological sample, comprising: (a) obtaining a biological sample from a subject after said subject ingested or consumed, or should have ingested or consumed, a composition comprising at least one detectable consumable marker; and (b) detecting the presence, absence, or quantity of said one or more detectable consumable markers or a modified thereof in the biological sample.

In some embodiments, the identity of the detectable consumable marker(s) and/or metabolizable consumable marker(s), and/or the amounts of each marker in the composition, are not disclosed to the subject and/or testing agency.

There are several advantages to the methods and compositions provided herein, including preservation of the privacy of the individual to be tested, and the ability to verify the individual as the source of many different biological samples, i.e., to track any biological source to an individual in any context. For example, the privacy of the subject is not so invaded by observing the consumption of a composition as disclosed herein as by observing the collection of a biological sample, e.g., urine. Also, with the methods, compositions and kits provided herein, all biological samples may be marked. In a hospital setting, this allows the source of any biological sample isolated from a patient to be verified after the patient has consumed one or more compositions allowing for such marking.

In a further embodiment, the identification of certain markers can be disclosed to the subject and/or testing agency while the corresponding compounds are intentionally omitted from the subject compositions, and accordingly are not ingested by a subject, and therefore be expected to be absent. These are referred to as “omitted markers”. The purpose of these is to prevent subjects from obtaining clean urine samples and adding a cocktail of GRAS compounds that could be found in the urine to falsely pass a marker test. Having expected non-present markers makes this less likely to successfully fool the test. These omitted markers can, accordingly, function as an additional negative control or negative standard for a biological sample. In some embodiments, the omitted markers are rare or obscure detectable consumable markers, e.g., not generally found in foodstuffs and pharmaceuticals.

One application of this technology is to label bodily samples in the drug court or rehab center setting. In these settings, there may be a high percent of subjects who would test positive for drugs, and consequently, there may be a high percentage of subjects in this setting who will attempt to foil a drug test, e.g., by one of the above described methods.

In some embodiments, the methods disclosed herein can be used to detect tampering with a biological sample, wherein a subject attempts to destroy or alter the drugs (or modified forms thereof) in a urine sample in order to impede their detection, e.g., by adding bleach to a urine sample. The methods disclosed herein can detect such tampering because the detectable consumable markers disclosed herein that function as positive controls or positive standards would also become absent or reduced in quantity in a biological sample upon such tampering.

An additional application of this technology is to label bodily samples, e.g., valuable pathology samples, in the clinical setting. This allows an internal chemical code to exist for patients such that when samples are taken from subject patients there is a secondary system for confirming that the sample came from a given patient. This comes into play in valuable (e.g. surgical) samples should the labeling fail or be confused in handling.

### Compositions

Also contemplated for verifying the source of a biological sample are compositions consisting essentially of one or more detectable consumable markers, wherein the detectable consumable markers are non-toxic and generally safe for consumption. In some embodiments, the compositions consist essentially of one, two or more, three or more, four or more, five or more, six or more, seven or more, eight or more, nine or more, ten or more, fifteen or more, or twenty or more detectable consumable markers.

In some embodiments, the detectable consumable markers are GRAS compounds. In some embodiments, the compositions consist essentially of one, two or more, three or more, or four or more, five or more, six or more, seven or more, eight or more, nine or more, ten or more, fifteen or more, or twenty or more GRAS compounds. In some embodiments, the compositions consist essentially of three to six GRAS compounds. In some embodiments, the compositions consist essentially of three to five GRAS compounds. In some embodiments, the compositions consist essentially of three to four GRAS compounds. In some embodiments, at least one GRAS compound is metabolized after consumption or ingestion. In some embodiments, the GRAS compounds are not usually found in foodstuffs or pharmaceutical compositions.

In a further embodiment, the methods further comprise obtaining a biological sample from a subject a time after a subject ingested or consumed, or should have ingested or consumed, a composition as disclosed herein, wherein at least one detectable consumable marker is present in said biological sample in a detectable amount, if said composition was consumed.

The nature of the markers comprised in the composition to be consumed determines the dosage and the length of time that should lapse between ingestion or consumption of the composition and obtaining a biological sample from the subject. In some embodiments, the compositions disclosed herein are formulated so that each of the compounds is detectable in urine as soon as 10 minutes to about 2 hours after consumption up to 24 hours after consumption. In some embodiments, the compositions disclosed herein are formulated so that each of the compounds is provided as a dose that is detectable in urine within 2 hours, 1 hour, 30 minutes, 20 minutes, or 10 minutes. In some embodiments, the marker(s) are detectable as soon as fifteen minutes after consumption. In some embodiments, the marker(s) are detectable as soon as twenty minutes after consumption. In some embodiments, the marker(s) are detectable as soon as twenty five minutes after consumption. In some embodiments, the marker(s) are detectable as soon as one hour after consumption.

In some embodiments, the compositions are formulated with detectable consumable markers that can be detected in a biological sample days, weeks or months after ingestion of the composition. Such a long lasting formulation may be especially useful in a hospital setting. In some embodiments, the detectable consumable markers may be detected one day, two days, three days, four days, five days, six days, or seven days after ingestion. In some embodiments, the detectable consumable markers may be detected one week, two weeks, three weeks, four weeks, five weeks, six weeks, seven weeks, eight weeks, twelve weeks or more after ingestion. In some instances, the one or more detectable consumable markers is/are lipophilic compound(s).

In a preferred embodiment, the compositions disclosed herein are formulated for oral administration. The compositions may be incorporated into a tablet, capsule, dissolvable film, aerosol, powder or liquid. The compositions disclosed herein can be formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, a composition disclosed herein further comprises a pharmaceutically acceptable diluent(s), excipient(s), or carrier(s). In some embodiments, the pharmaceutical compositions includes other components typically included in pharmaceutical compositions, such as preserving, stabilizing, wetting or emulsifying agents, solution promoters, salts for regulating the osmotic pressure, and/or buffers.

In certain embodiments, a composition disclosed herein is administered orally to a subject. In some embodiments, a composition disclosed herein is administered orally to a subject in the form of an oral solution. In some embodiments, a composition disclosed herein is administered orally to a subject in the form of an oral unit capable of being swallowed, i.e., a dosage form. In some embodiments, a composition described herein is in unit dosage forms suitable for single administration of precise amounts of detectable consumable markers.

### Analysis

In some embodiments, the presence or absence of a marker is detected using any well-known immunoassay or chromatographic technique. In some embodiments, the presence or absence of a marker is detected with an antibody specific for said marker. In some embodiments, the presence of absence of a marker may be detected using fluorescence, UV emission, or IP spectroscopy.

There are a variety of immunoassay formats known to those of ordinary skill to detect a marker in a sample using an antibody specific for the marker. See, e.g., Harlow and Lane, Antibodies: A Laboratory Manual, Cold Spring Harbor Laboratory, 1988. Non-limiting examples include immunoprecipitation, ELISA, Western blot analysis, immunohistochemistry, immunofluorescence, “sandwich” immunoassays, immunoradiometric assays, gel diffusion precipitation reactions, immunodiffusion assays, in situ immunoassays, precipitation reactions, agglutination assays, complement fixation assays, protein A assays, immunoelectrophoresis assays, radioimmunoassay, a strip test, a point of care test, and the like.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated.

In some embodiments, the assays involve the use of at least one antibody immobilized on a solid support to specifically bind to and capture markers from the sample. The bound markers may then be detected using a detection reagent that contains a reporter group and specifically binds to the antibody/marker complex.

The solid support may be any material known to those of ordinary skill in the art. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The antibody thereof may be immobilized on the solid support using a variety of techniques known to those of skill in the art, which are amply described in the patent and scientific literature. In the context of the present invention, the term “immobilization” refers to both non-covalent association, such as adsorption, and covalent attachment (which may be a direct linkage between the antibody and functional groups on the support or may be a linkage by way of a cross-linking agent). Immobilization by adsorption to a well in a microtiter plate or to a membrane is preferred. In such cases, adsorption may be achieved by contacting the antibody, in a suitable buffer, with the solid support for a suitable amount of time. The contact time varies with temperature, but is typically between about 1 hour and about 1 day.

In some embodiments, the assay to detect the presence or absence or quantity of a detectable consumable marker is accomplished using a flow-through, test strip or “dipstick” type apparatus. The apparatus may comprise a porous carrier, a labeled specific binding reagent for a detectable consumable marker which labeled specific binding reagent is freely mobile within the porous carrier when in the moist state, but is placed on the porous carrier in a “first zone” or “reaction zone,” and unlabelled specific binding reagent for the same detectable consumable marker which unlabelled reagent is permanently immobilized in a “detection zone” on the carrier material and is therefore not mobile in the moist state, the relative positioning of the labeled reagent and detection zone being such that liquid biological sample applied to the device can pick up labeled binding reagent and thereafter permeate into the detection zone. In some embodiments, the carrier material is nitrocellulose and the specific binding reagents are immunoglobulins. The apparatus is contacted with a liquid biological sample for analysis such that the sample permeates by capillary action through the porous carrier material via the reaction zone into the detection zone and the labeled reagent migrates therewith from the reaction zone to the detection zone, the presence of analyte in the sample being determined by observing the extent (if any) to which the labeled reagent becomes bound in the detection zone. The labeled reagent, the detectable consumable marker (if present) and the immobilized unlabelled specific binding reagent cooperate together in a “sandwich” reaction. This results in the labeled reagent being bound in the detection zone if analyte is present in the sample. The two binding reagents must have specificities for different epitopes on the analyte. In some embodiments, a “control” zone can be included in the apparatus to convey an unrelated signal to the user that the device has worked. For example, the control zone can be loaded with an antibody that will bind to the labeled antibody from the reaction zone, e.g. an “anti-mouse” antibody if the labeled body is one that has been derived using a murine hybridoma, to confirm that the sample has permeated the test strip. Alternatively, the control zone can contain an anhydrous reagent that, when moistened, produces a color change or color formation, e.g. anhydrous copper sulphate which will turn blue when moistened by an aqueous sample. As a further alternative, a control zone could contain immobilized analyte which will react with excess labeled reagent from the reaction zone. As the purpose of the control zone is to indicate to the user that the test has been completed, the control zone should be located downstream from the detection zone in which the desired test result is recorded. A positive control indicator therefore tells the user that the sample has permeated the required distance through the test device.

Other methods can be used to quantitatively measure the levels of the marker. For example, in some embodiments, an antibody reacts with a marker in a liquid phase, and the immune complexes are quantitatively measured by an immunoprecipitation technique. In some embodiments, an anti-marker antibody or fragment thereof is detectably labeled (e.g., with an isotope or an enzyme). In some embodiments, the detectable antibody is added directly to a liquid biological sample (e.g., urine) to form immune complexes. In some embodiments, the immune complexes are precipitated with polyethylene glycol. In some embodiments, the immune complexes are isolated with a secondary antibody (e.g., goat anti-human immunoglobulin) or other kind of binding molecules (e.g., protein A or protein G) that is bound to a solid support (e.g., agarose or sepharose beads). In some embodiments, the immunoprecipitates are washed several times after being separated from the liquid sample and examined for intensity of the detectable label (e.g., radioactivity). Any marker present in the sample can thus be detected and quantified. Optionally, in some embodiments, an unlabelled antibody can also be added to compete with the labeled antibody for binding to marker.

Analysis may also occur via a computer readable medium. Accordingly, also disclosed herein is a system comprising a database that provides information on the contents (i.e., the makeup of the composition(s) and links that information to specific bar codes). Such database can be part of a specific computer comprising a computer readable medium. The computer is programmed to specifically perform one or more of the following tasks: (1) input barcode info; (2) link a barcode with contents of a composition; (3) confirm authenticity of the source of a biological sample; (4) provide a report; (5) transmit any of the above over the internet; (6) input data from screening assays; and/or (7) track authenticity from a single source over a period of time. In some embodiments, data regarding the presence, absence, or quantity of specific detectable consumable markers is sent to a computer, which corresponds said data with compositions and a list of the contents of the composition. In some embodiments, the computer compares the data to each other. In some embodiments, the computer makes a determination of whether the biological sample submitted by a subject matched the post-consumption profile of a given composition. In some embodiments, said data, or determination, is forward to a remote user.

### Kits

Disclosed herein are kits comprising one or more compositions of the invention. In some embodiments, each composition is packaged, e.g., in a kit, along with a means for correlating the test results with the identity of the composition, i.e., a list of the consumable markers in the composition. For example, in some embodiments, the kits further comprise a barcode associated with the composition, i.e., the composition is packaged with coded information (e.g., number, letter string, a bar code, an RFID tag, etc.) that informs the tester of the exact nature of the composition packaged therein. In some embodiments, an infinite number of barcodes can be generated to identify a more limited amount of compositions according to the invention. In some embodiments, the kit further comprises additional consumable compositions of the invention, each with a different barcode. In some embodiments, the kit comprises duplicate barcodes for each composition, wherein one barcode is given to the subject to identify his biological sample submission, and another barcode is retained by the testing agency to identify the post-consumption expected profile of the composition. Another means for correlating the test results includes a label that provides the nature of the composition, wherein the label has a tamper resistant cover.

In some embodiments, the kit further comprises a collection device designed for the collection and/or storage of a particular biological sample. In some embodiments, the kit comprises a means for correlating the results obtained with the biological sample to the makeup of the composition. In some embodiments, the kit further comprises reagents and components for detecting the present or absence of detectable consumable markers in the biological sample. In some embodiments, the kits also include pre-printed forms that provide instructions regarding the consumption of the composition, to assist in the documentation of the consumption of markers, and/or the return of the sample collected.

With any given set of detectable consumable markers there is a binary quality to each marker: it can be expected to be present or absent. For example if there are five different markers there are two to the fifth power (32) possible expected results. Furthermore one can periodically change the markers and corresponding kits and/or detection systems to stay ahead of those who may try to undermine the system. Thus, the number of different markers and corresponding detection sets is essentially infinite. This makes foiling the system very difficult.

### Example 1

### Determination of Dose Amount of a Detectable Consumable Marker

At least 10 average men and/or women volunteers are assigned to consume a particular detectable consumable marker. Prior to consumption of the detectable consumable marker, the volunteers are requested to void and urine sample are retained to measure the baseline amounts of the marker. The volunteers are each given a composition comprising a different amount of marker either in solution or with a beverage. The volunteers are then requested to void again and provide their urine samples, which are assayed for the presence of the marker. The minimum amount of the marker which generates a clear detectable signal in the urine (as compared to baseline) may serve as an appropriate amount of marker to include in a composition as disclosed herein.

### Example 2

### Administration of Detectable Consumable Markers

In one exemplary embodiment, inulin, amyris and eugenol are provided as detectable markers and alpha amylase is included as a metabolizable marker in the consumable marker composition administered to the subject. A urine sample is subsequently obtained from the subject and screened for the presence or absence of each of these compounds. If the urine sample originated from the subject, the urine sample is expected to have detectable levels of inulin, amyris, and eugenol, but not have detectable levels of alpha amylase.

### Example 3

### Administration of Composition with Omitted Markers

In a further embodiment, the screening assay of Example 2 is further modified to include a component list listing components that are not included in the compositions, i.e., are not actually found in the consumable marker composition administered to the patient. Musk ambret and Canaga can be included as omitted markers, and if found during sample screening it is likely that the subject attempted to anticipate and fool the test by adding detectable consumable marker compounds to a clean urine sample.

### Example 4

### Testing of a Biological Sample for Omitted Markers

A urine sample is obtained from a subject administered the detectable consumable markers described in Example 2. The urine sample is assayed for the detectable consumable markers described in Example 2, and also for at least one obscure or rare detectable consumable marker, i.e., an “omitted marker,” that was not administered. If the sample is found positive for the at least one obscure or rare detectable consumable marker, the subject may have attempted to fool the test by adding detectable consumable markers to a clean urine sample.

### Example 5

### Use of a Kit According to the Invention

A kit according to the invention includes “dipstick” style testing strips for six different detectable consumable markers, wherein each dipstick has separate reaction, testing and control zones. The kit also includes a packet comprising a composition according to the invention and a barcode identifying the contents of the composition, and the expected post-consumption detection profile. After a subject consumes the composition and provides a urine sample twenty minutes post-consumption, the biological sample is placed in a container. The test strips are dipped into the container containing the biological sample. The presence or absence of a positive signal for any of the six different detectable consumable markers is correlated with the expected post-consumption profile, wherein a match verifies the subject as the source of the biological sample.

### Example 6

### Use of a System According to the Invention

A subject goes to a laboratory for drug testing and is given a composition according to the invention, which the subject consumes. The composition packaging is tagged with a barcode identifying the contents of the composition and its expected post-consumption profile. The subject then gives a urine sample to the laboratory within an hour of consuming the composition. The laboratory runs the urine sample through an apparatus which detects the presence, absence or quantity of the detectable consumable markers identified by the barcode. The apparatus send this information to a computer, which compared this information with the barcoded information. The computer subsequently makes a determination regarding whether the profile of the detectable consumable markers in the urine sample matches the expected post-consumption profile. The computer sends this information over the internet to an interested individual or agency.
While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1
Amprolium (1-(4-amino-2-n-propyl-5-pyrimldinyl-methyl)-2-picolinium chloride hydrochloride-
http://sitem.herts.ac.uk/aeru/vsdb/Reports/1747.htm (may be active antibiotic)
Amyris - FL/ADJ, REG, GMP, Used in conjunction w/flavors - 172.5100-
http://www.bojensen.net/EssentialOilsEng/EssentialOils02/EssentialOils02.htm
eugenol clove leaf oil metabolized to Glucuronide and sulfate conjugates were identified in urine.
J Vet Pharmacol Ther. 2006 August; 29(4): 265-70., Pharmacokinetics and anesthetic activity of
eugenol in male Sprague-Dawley rats., Guenette S A, Beaudry F, Marier J F, Vachon P., Source,
Department of Veterinary Biomedicine, Faculty of Veterinary Medicine, University of Montreal, St
Hyacinthe, Canadahttp://www.marinwater.org/documents/Chap6 CloveOil 8 28 08.pdf
Adipic Acid: J Chromatogr Sci. 1985 September; 23(9): 407-10. Direct determination of adipic acid in
urine by extractive alkylation. Adinolfe N A, Bicking M K.
Dilauryl thiodipropionate (DLTDP), Ingested DLTDP was excreted in the urine as thiodipropionic
acid: Int J Toxicol. 2010 July; 29(4 Suppl): 137S-50S., Final safety assessment of thiodipropionic acid
and its dialkyl esters as used in cosmetics. Diamante C, Fiume M Z, Bergfeld W F, Belsito D V, Hill
R A, Klaassen C D, Liebler D C, Marks J G Jr, Shank R C, Slaga T J, Snyder P W, Alan Andersen F.
Source, Cosmetic Ingredient Review, Washington, DC 20036, USA.
Ethyl formate: Int Arch Occup Environ Health. 1999 July; 72(4): 215-22., Determination of
N-nitrosodiethanolamine in urine by gas chromatography thermal energy analysis: application in
workers exposed to aqueous metalworking fluids., Ducos P, Gaudin R, Francin J M.
Ethyl methylphenylglycidate: Food Cosmet Toxicol. 1981 December; 19(6): 691-9., Long-term toxicity
study of ethyl methylphenylglycidate (strawberry aldehyde) in the rat., Dunnington D, Butterworth
K R, Gaunt I F, Mason P L, Evans J G, Gangolli S D.
Formic acid (x-ref-Ethyl formate)
Geraniol (3,7-dimethyl-2,6 and 3,6-octadien-1-ol): Xenobiotica. 1984 May; 14(5): 365-74.,
Metabolism of geraniol and linalool in the rat and effects on liver and lung microsomal
enzymes., Chadha A, Madyastha K M.
Geranyl acetate (geraniol acetate): Br J Pharmacol. 2006 March; 147(5): 476-85., Interspecies
pharmacokinetics and in vitro metabolism of SQ109., Jia L, Noker P E, Coward L, Gorman G S,
Protopopova M, Tomaszewski J E., Developmental Therapeutics Program, National Cancer Institute,
NIH, 6130 Executive Blvd., Rm 8042, Rockville, MD 20852, USA. jiale@mail.nih.gov
Glucono delta-lactone: J Clin Chem Clin Biochem. 1979 April; 17(4): 257-67., [Investigations on the
utilization of D-gluconate and D-glucono-delta-lactone in the metabolism of the normal and alloxan
diabetic rat (author's transl)], Tharandt L, Hübner W, Hollmann S.
Glycoryrrhiza: metabolized to pentafluorobenzyl-ester/trimethylsilyl-ether, J Chromatogr B
Biomed Sci Appl. 1999 Aug 20; 731(2): 323-34., Determination of urinary 18 beta glycyrrhetinic acid
by gas chromatography and its clinical application in man., Guillaume C P, van der Molen J C,
Kerstens M N, Dullaart R P, Wolthers B G., Department of Clinical Chemistry, University Hospital
Groningen, The Netherlands.
3,5-Diiodosalycylic acid: Med Chem. 2007 November; 3(6): 546-50. A salicylic acid-based analogue
discovered from virtual screening as a potent inhibitor of human 20alpha-hydroxysteroid
dehydrogenase. Dhagat U, Carbone V, Chung R P, Matsunaga T, Endo S, Hara A, El-Kabbani O.
Musk ambrette: Toxicol Lett. 2002 May 28; 131930: 147-51., Dermal absorption and disposition of
musk ambrette, musk ketone and musk xylene in human subjects., Hawkins D R, Elsom L F,
Kirkpatrick D, Ford R A, Api A M.
n-Butoxypolyoxyethylene polyoxypropylene glycol: Oral toxicity and excretion of four
commercial polyoxyalkylene glycol compounds, H. F. Smyth, Jr., C. S. Weil, J. M. King¹, J. B.
Knaak², L. J. Sullivan and C. P. Carpenter Carnegie-Mellon University, Mellon Institute, Pittsburgh,
Pennsylvania 15213, USA, Received 18 Jun. 1969. Available online 27 Sep. 2004.
Canaga (ylang ylang) methyl benzoate, 4-methylanisole, and benzyl benzoate - benzyl benzoate are
metabolites http://www.aseanfood.info/Articles/11022798.pdf
Caraway (corvone) multiple testable metabolites
http://www.inchem.org/documents/jecfa/jecmono/v042je18.htm
Hyssop Antioxidant-rich spice added to hamburger meat during cooking results in reduced meat,
plasma, and urine malondialdehyde concentrations., Li Z, Henning S M, Zhang Y, Zerlin A, Li L,
Gao K, Lee R P, Karp H, Thames G, Bowerman S, Heber D., Am J Clin Nutr. 2010 May; 91(5): 1180-
4. Epub 2010 Mar. 24. PMID: 20335545 [PubMed - indexed for MEDLINE]
Immortelle: Molecules. 2008 Apr. 7; 13(4): 795-803., Contribution to the analysis of the essential oil
of Helichrysum italicum (Roth) G. Don. Determination of ester bonded acids and phenols.,
Mastelić J, Politeo O, Jerković I. Source: Department of Organic Chemistry, Faculty of Chemistry
and Technology, N. Tesle 10/V, 21000 Split, Croatia. josip.mastelic@ktf-split.hr
Linden flowers: Metabolized to quercetin. SPE-HPLC method for the determination of four
flavonols in rat plasma and urine after oral administration of Abelmoschus manihot extract, Xianyin
Lai^{a}, Yuying Zhao^{a}, Hong Liang, ^{a,}, Yanjing Bai^{a}, Bin Wang^{a} and Dean Guo, ^{a,}, ^{a}Department of
Natural Medicines and the State Key Laboratory of Natural and Biomimetic Drugs, School of
Pharmaceutical Sciences, Peking University Health Science Centre, Beijing 100083, PR China,
Received 8 Oct. 2006; accepted 31 Dec. 2006. Available online 10 Jan. 2007.
Lovage: metabolized to 4,5-dimethyl-3-hydroxy-2[5H]furanone (sotolone), J Inherit Metab Dis.
1999 April; 22(2): 107-14., 4,5-dimethyl-3-hydroxy-2[5H]furanone (sotolone)—the odour of maple
syrup urine disease., Podebrad F, Heil M, Reichert S, Mosandl A, Sewell A C, Böhles H., Institute of
Food Chemistry, University of Frankfurt, Frankfurt am Main, Germany.
Maidenhair/ginko: Metabolized to quercetin and kaempferol, Rapid Commun Mass Spectrom.
1998; 12(4): 153-6., Analysis of flavonoids in tablets and urine by gas chromatography/mass
spectrometry and liquid chromatography/mass spectrometry., Watson D G, Pitt A R., Department of
Pharmaceutical Sciences, University of Strathclyde, Glasgow, UK.
Naringin: Yao Xue Xue Bao. 2010 June; 45(6): 761-6., [UPLC-Q-TOF/MS analysis of naringin and
naringenin and its metabolites in rat urine and feces after intragastric administration of alcohol
extract of Exocarpium Citri grandis]., Sun G L, Qian D W, Duan J A, Li X M, Wan Y., Nanjing
University of Chinese Medicine, Nanjing 210046, China.
n-Octanoic (caprylic) acid: Scand J Gastroenterol. 2009: 44(9): 1067-75., Desirable
pharmacokinetic properties of (13)C-uracil as a breath test probe of gastric emptying in comparison
with (13)C-acetate and (13)C-octanoate in rats., Sugiyama E, Inada M. Kunizaki J, Tobita K.
Yoshida T. Kashimoto M, Hirao Y, Sato H.
Opopanax: Metabolized to peucelinenoxide acetate (should be a unique source. Fitoterapia. 2005
December: 76(7-8): 768-70. Epub 2005 Oct.17., A new irregular diterpenoid from Opopanax chironium.,
Muckensturm B, Boulanger A. Ouahabi S, Reduron JP.
Pennyroyal: J Toxicol Environ Health A. 2007 September: 70(17): 1416-23., 14C-labeled pulegone and
metabolites binding to alpha2u-globulin in kidneys of male F-344 rats., Ferguson L J, Lebetkin E H,
Lih F B, Tomer K B, Parkinson H D, Borghoff A, Burka L T.
Pipsissewa leaves: LC-MS method for determination and pharmacokinetic study of chimaphilin in
rat plasma after oral administration of the traditional Chinese medicinal preparation Lu xian cao
decoction., Zhang Y, Chen X, Oin S, Kim C, Teb
St. Johnswort: J Chromatogr A. 2005 Nov. 4: 1093(1-2): 1-10. Epub 2005 Aug.16., Single-drop
liquid-phase microextraction for the determination of hypericin, pseudohypericin and hyperforin in
biological fluids by high performance liquid chromatography., Gioti E M, Skalkos D C, Fiamegos
Y C, Stalikas C D.
Serpentaria (Virginia snakeroot): major component aristolochic acid, Biomed Chromatogr. 2010
December: 24(12): 1350-5. doi: 10.1002/bmc.1448.; Determination of aristolochic acid in urine using
hollow fiber liquid-phase microextraction combined with high-performance liquid chromatography.,
Yang Y, Chen J, Shi Y P.
Tannic acid: metabolized to 4-0-Methylgallic acid (4-OMGA), pyrogallol (PY), and resorcinol
(RE), J Agric Food Chem. 2003 Jan. 1: 51(1): 331-9., Method for analysis of tannic acid and its
metabolites in biological samples: application to tannic acid metabolism in the rat., Nakamura Y,
Tsuji S, Tonogai Y.
Tuberose: Natl Toxicol Program Tech Rep Ser. 1986 August : 250: 1-204., NTP Toxicology and
Carcinogenesis Studies of Benzyl Acetate (CAS No. 140-11-4) in F344/N Rats and B6C3F1 Mice
(Gavage Studies)., National Toxicology Program.
Zedoary: metabolized to curcumin, Indian J Med Res. 2010 May; 131: 682-91., Tissue distribution &
elimination of capsaicin, piperine & curcumin following oral intake in rats., Suresh D, Srinivasan K.
Source: Department of Biochemistry & Nutrition, Central Food Technological Research Institute
(CSIR), Mysore, India.

TABLE 2
Alpha-amylase - ENZ, REG, used to modify food starch -172.892
Aminoglycoside 3′phosphotransferase II - 173.170
Amylase from Aspercillgus Oryzae - ENZ, REG -137.105, 137.155, 137.160, 137.165, 137.170,
137.175, 137.180, 137.185, 137.200, 137.205
gum karaya: Food Addit Contam. 1985 January-March; 2(1): 33-6., The absence of rhamnose in human
urine following the ingestion of gum karaya (Sterculia). Anderson A W, Brydon W J, Eastwood M A,
McDougall F J, Anderson D M
Guarana: Z Lebensm Unters Forsch. 1996 July; 203(1): 95-8., Studies on the essential oil from
guarana., Benoni H, Dallakian P, Taraz K., Institut für Organische Chemie der Universität Köln,
Köln, Germany.
Papain: protein
Sterculia gum (karaya gum): Food Addit Contam. 1985 January-March; 2(1): 33-6.
The absence of rhamnose in human urine following the ingestion of gum karaya (Sterculia).
Anderson A W, Brydon W J, Eastwood M A, McDougall F J, Anderson D M.

What is claimed is:

1. A method of verifying a source of a biological sample, comprising:
(a) obtaining the biological sample from a subject after the subject consumed, or should have consumed, a composition comprising at least one detectable consumable marker; and
(b) detecting the presence, absence, or quantity of said one or more detectable consumable markers or a modified form thereof in the biological sample.

2. The method of claim 1 further comprising verifying whether the subject is the source of the biological sample by comparing results detected in step (b) with an expected post-consumption profile in the biological sample.

3. The method of claim 1 wherein the biological sample is urine.

4. The method of claim 1, wherein the obtaining is performed after a time sufficient for the detectable consumable marker or modified form thereof to be detectable in the biological sample.

5. The method of claim 1, wherein the obtaining is performed after less than 2 hours, 1 hour, 30 minutes, 20 minutes, or 15 minutes from the time the composition was consumed or should have been consumed.

6. The method of claim 1, wherein the composition comprises at least 2, 3, 4, 5, 6, or 7 detectable consumable markers.

7. The method of claim 1, wherein at least one of the detectable consumable markers is detected in the biological sample in its parental (unmodified) form.

8. The method of claim 1, wherein at least one of the detectable consumable markers is detected in the biological sample in the modified form.

9. The method of claim 1, further comprising comparing the presence or absence of the set of detectable consumable markers in the biological sample to an expected post-consumption profile of said set of detectable consumable markers in the biological sample, wherein a match between the presence, absence, or quantity of the detectable consumable markers or modified forms thereof in the biological sample and an expected post-consumption profile is indicative that the subject is the source of the biological sample, and optionally a second assay is performed on the biological sample.

10. The method of claim 9, wherein the second biological assay is selected from the group consisting of prenatal testing, pregnancy testing, drug testing, hormone testing, or liver function testing.

11. The method according to any one of the preceding claims, further comprising assigning the at least one detectable consumable marker to the subject prior to the obtaining step.

12. The method according to any one of the preceding claims, further comprising determining the baseline level of the at least one detectable consumable marker in a biological sample from the subject, prior to obtaining said biological sample.

13. The method according to any one of the preceding claims, further comprising observing the subject consume or ingest the composition.

14. The method according to any one of the preceding claims, wherein the presence, absence or quantity of said at least one detectable consumable marker is detected with an antibody specific for the at least one detectable consumable marker.

15. The method of claim 14, wherein the antibody specific for the at least one detectable consumable marker is attached to a solid support.

16. The method according to any of claims 1-13, wherein the presence, absence, or quantity of said at least one detectable consumable marker is detected using chromatographic separation.

17. A consumable composition consisting essentially of two or more detectable consumable markers, wherein the detectable consumable markers are non-toxic and generally safe for consumption.

18. The composition of claim 17, wherein the detectable consumable markers are Generally Regarded as Safe (GRAS) compounds.

19. A consumable composition comprising three to six GRAS compounds.

20. A consumable composition according to claim 19, comprising three to five GRAS compounds.

21. A consumable composition according to claim 20, comprising three to four GRAS compounds.

22. The composition of claim any of claims 17-21, wherein the composition is formulated for oral administration.

23. The composition of any of claims 18-22, wherein each of the detectable consumable markers is provided at a dose detectable in urine within 2 hours.

24. The composition of any of claims 18-23, wherein at least one of the GRAS compounds is metabolized after consumption or ingestion.

25. The composition of any of claims 18-24, wherein the GRAS compounds are not generally found in foodstuffs or pharmaceuticals.

26. The composition of any of claims 18-25, wherein the GRAS compound is selected from the list of compounds of Table 1 or Table 2.

27. The composition of claim 26, comprising three to six compounds from Table 1.

28. The composition of claim 27, further comprising at least one compound from Table 2.

29. The composition of claim 26, wherein the GRAS compound is selected from: dilauryl thiodipropionate; ethyl formate; ethyl methylphenylglycidate; formic acid; geraniol; geraniol acetate; glucono delta-lactone; glycoryrrhiza; 3,5-diiodosalycylic acid; musk ambrette; n-Butoxypolyoxyethylene polyoxypropylene glycol; canaga; caraway; hyssop; immortelle; linden flowers; lovage; maidenhair/gingko; naringin; n-Octanoic acid; opopanax; pennyroyal; pipsissewa leaves; St. John's Wort; serpentaria; tannic acid; tuberose; zedoary; alpha-amylase; aminoglycoside 3′phosphotransferase II; amylase from aspercillgus oryzae; gum karaya; guarana; papain; sterculia gum; and sterculia gum.

30. A kit comprising the composition any of claims 17-29.

31. The kit of claim 25, wherein the kit further comprises a list of detectable consumable markers in said composition.

32. The kit of claim 25, wherein the kit further comprises a barcode associated with the composition.

33. The kit of claim 27, further comprising additional consumable compositions, optionally, each with a different combination of detectable consumable markers.

34. A system comprising a database linking one or more unique barcodes with a unique set of detectable consumable markers or detectable modified forms thereof.

35. A system according to claim 34, wherein said database is located inside a computer.

36. A system according to claim 35, wherein said computer is linked to the internet.

37. A system according to claim 36, wherein said computer is capable of sending information to a remote location.

38. A system according to claim 34, wherein the computer is programmed to specifically perform one or more of the following tasks: (1) input barcode info; (2) link a barcode with contents of a composition; (3) confirm authenticity of the source of a biological sample; (4) provide a report; (5) transmit any of the above over the internet; (6) input data from screening assays; and/or (7) track authenticity from a single source over a period of time.
